(19) United States
(12) Patent Application Publication
(10) Pub. No.: US 2026/0331291 A1
(43) Pub. Date: Oct. 22, 2026

(54) Orchestrix Integrated Clinical AI Orchestration System

(71) Applicant: Jennifer Joiner Bryan, Brandon, MS (US)

(72) Inventor: Jennifer Joiner Bryan, Brandon, MS (US)

(21) Appl. No.: 19/185,913

(22) Filed: Apr. 22, 2025

(51) Int. Cl. G16H 50/30; G06Q 20/10

(52) U.S. Cl. CPC G16H 50/30; G06Q 20/102

(57) ABSTRACT

A clinical orchestration system and method for governing artificial intelligence (AI)-assisted decision-making through symbolic fallback logic, credentialed invocation thresholds, and traceable authorship enforcement. The system coordinates patient data, electronic health record (EHR) inputs, and AI outputs using shimmer-governed suppression, override gating, and audit-trace documentation controls. Invocation is permitted only when shimmer thresholds are satisfied, and authorship is credential-verified. Fallback logic is triggered in pediatric, psychiatric, or epistemically unstable contexts, with override gated by credential tier and shimmer-authenticated timestamp. All invocation attempts are trace-logged and exportable. Symbolic rendering may be suppressed, delayed, or rerouted based on shimmer policy profiles. The system includes tiered endpoint licensing, override revocation, and shimmer token replay for payer or legal audits. Symbolic output is blocked unless shimmer-token trace, authorship continuity, and credential match conditions are met. The invention ensures trace-based AI orchestration in regulated clinical environments, preserving authorship integrity and fallback-author governance.

This application is a Continuation-in-Part of U.S. patent application Ser. No. 19/170,774, titled “AEONFRAME: Symbolic Cognitive Interface and Fallback Processing Architecture,” filed Apr. 4, 2025, which is hereby incorporated by reference in its entirety.

A clinical orchestration system is disclosed that coordinates predictive analytics, real-time recommendation gating, ethical fallback, billing automation, and memory continuity through a rule-based symbolic invocation architecture. The system integrates multiple AI modules under shimmer-authored safeguards including traceable consent logic, credential-bound invocation, outcome attribution, and cognitive state protection. A central orchestration engine manages workflow across safety gating, documentation generation, and closed-loop learning, while preserving symbolic context across sessions through tethered memory continuity. Modular licensing enforcement and emotional override mechanisms ensure only traceable, compliant actions reach clinicians. The system supports deployment in jurisdictionally constrained environments, complies with HIPAA, EU AI Act, and WHO ethical frameworks, and includes shimmer protocol interoperability with ShimmerSafe, ShimmerFrame, and Invokea. Government-only fallback deployment and CPT/SNOMED-free export controls are included. The invention solves integration, authorship, and regulatory traceability gaps in clinical AI by providing a unified, auditable, and ethically constrained orchestration platform.

### FIELD OF THE INVENTION

This invention relates to computer-implemented clinical decision support and healthcare workflow automation. In particular, it concerns an integrated system for orchestrating multiple artificial intelligence modules, including predictive analytics, clinical recommendation, safety gating, outcome tracking, memory continuity, and administrative automation, under a unified symbolic invocation architecture in a healthcare environment.

### BACKGROUND

Healthcare delivery increasingly leverages artificial intelligence (AI) and automated decision support tools. However, conventional solutions tend to address isolated workflow aspects rather than providing a comprehensive, coordinated approach. For example, some existing systems focus solely on generating risk scores or problem lists from electronic health records (EHRs), others deliver basic alerts or recommendations to clinicians without integrated follow-up, and separate computer-assisted coding tools automate billing by analyzing clinical notes post hoc. These siloed systems operate independently, leading to inefficiencies and potential safety gaps:

Fragmented Decision Support: A predictive model might flag a patient at high risk, but its output often remains disconnected from the point-of-care action. Clinicians receive risk scores or lengthy problem lists that are not directly translated into recommended interventions. This lack of integration means the burden falls on the provider to interpret predictions and decide on actions, which can delay critical interventions.

Lack of Safety Gating: Traditional clinical decision support alerts (e.g., drug allergy checks in EHRs) operate in isolation and are not seamlessly combined with AI-generated recommendations. As a result, AI provisions may reach clinicians without rigorous vetting against safety rules, leading to alert fatigue from separate systems or, worse, unsafe recommendations being presented.

No Closed-Loop Learning: Most current systems do not learn from patient outcomes. After an alert or recommendation is given, the system rarely tracks what happened to the patient. This omission fails to improve future recommendations or provide accountability for AI-driven decisions. Consequently, the AI does not adapt based on real-world efficacy of its provisions.

Administrative Overheads: Billing and documentation often remain manual or handled by separate natural language processing tools that parse clinician notes after the fact. Such approaches, exemplified by some existing coding products, operate retrospectively and in silos, which can introduce errors or omissions. They also duplicate effort: clinicians must document actions, and then a separate system translates that into billing codes, sometimes requiring human review.

Context Discontinuity: Furthermore, AI interactions with clinicians or patients typically treat each session independently. There is little to no persistence of context or “memory” of prior sessions' tone and content. For instance, a virtual assistant that interacted with a patient last week may not recall that patient's preferences or emotional state in the next session, leading to redundant questions or inconsistent personality in the AI's responses. This lack of continuity can erode user trust and engagement.

Consent and Ethical Constraints: Existing AI solutions often lack mechanisms to ensure that actions are taken only with proper patient consent or in alignment with ethical guidelines. For example, an AI module might automatically contact a patient's family based on a risk alert without a clear check for the patient's consent to share information. Similarly, purely algorithmic systems may suggest interventions that, while medically relevant, conflict with a patient's expressed values or institutional ethics policies, simply because those considerations aren't encoded.

Overall, the absence of a unified orchestration leads to inefficient, and sometimes unsafe, healthcare workflows. Clinicians are overwhelmed by disjointed alerts, patients may face AI interactions that feel impersonal or inconsistent, and healthcare providers miss opportunities to learn from outcomes or streamline administrative tasks. There is a clear need for a comprehensive platform that integrates predictive analytics with decision recommendation, safety checks, consent management, continuous learning, and automated follow-through actions in one coordinated system. Such an integrated solution must be designed to avoid overlap with known prior technologies (to ensure freedom-to-operate globally) while maximizing patient safety, ethical compliance, and practical utility.

### SUMMARY OF THE INVENTION

Overview: The present invention provides “Orchestrix,” an integrated clinical AI orchestration platform that unifies multiple functional modules into a single, coordinated system. Orchestrix expands upon the disclosure of the original AeonFrame patent (U.S. application Ser. No. 19/170,774) by incorporating new components and interactions that greatly enhance safety, continuity, and automation. The system is built around a Symbolic Invocation Architecture (SIA), a rule-driven orchestration engine that governs when and how each AI module is activated, ensuring all actions comply with safety rules, ethical guidelines, and consent requirements, with fallback mechanisms for reliability. This CIP strengthens the legal defensibility and global patentability of the platform by explicitly integrating these novel features and structuring claims to require the unique combination, thereby distinguishing Orchestrix from prior art held by major industry players.

Key Integrated Components: In one embodiment (illustrated in FIG. 1), Orchestrix comprises the following interlinked modules working in concert:

Predictive Analytics Module, for example, AeonFrame, which analyzes patient data (e.g. EHR data, vital signs, historical outcomes) to generate real-time risk scores or outcome predictions. Unlike prior systems that stop at producing a static risk assessment or problem list, this module's output is immediately used to drive downstream decision-making within Orchestrix's workflow.

Intervention Recommendation Module (Clinical Interface), a decision support interface that formulates patient-specific intervention recommendations based on the predictive insights. It presents suggested actions (diagnostic or therapeutic) to the clinician in real time. Crucially, these recommendations are only displayed after passing through the safety and compliance checks of the SIA and ShimmerSafe gating (see below). This multi-layered logic ensures the recommendations are context-aware and filtered for safety, distinguishing them from simpler AI provision tools that lack such gating (e.g., basic alert systems or ranking engines). The module includes a user interface for clinicians to review provisions and provide input or approval.

Safety Gating Module, for example, ShimmerSafe, which serves as a real-time safety and compliance filter for all proposed interventions. ShimmerSafe automatically checks each candidate recommendation against medical safety rules, clinical protocols, patient-specific constraints, and “shimmer” metrics (defined below) before it reaches the clinician. Recommendations that conflict with drug interaction warnings, contraindications, institutional policies, or other safety criteria are flagged or modified before being shown. ShimmerSafe's shimmer metrics are fine-grained physiologic and cognitive indicators (such as subtle variations in heart rate or blood pressure, fetal heart rate variability, changes in a patient's responsiveness or mood) that provide early warnings of risk in sensitive populations. For example, in a maternal-fetal context, ShimmerSafe can monitor real-time fetal heart rate variability and maternal vital trends; if a dangerous pattern (“shimmer”) is detected, it can gate or adjust any recommended intervention (like delaying a suggested medication that could exacerbate stress) and immediately alert providers. In pediatric or cognitive health scenarios, shimmer metrics might include a child's micro-behaviors or a patient's speech latency as signs of cognitive decline, allowing the system to proactively ensure safety. This integrated safety gating is a critical differentiator: whereas typical EHR systems might perform basic checks (e.g., allergy alerts) in isolation, Orchestrix's safety module is embedded within the AI recommendation loop, making it an essential gate that must be passed for any AI-generated provision to be delivered. This not only protects patients but also ensures that any similar system would need to incorporate an equivalent safety layer to achieve the same functionality.

Memory Continuity Module, a context persistence system that stores symbolic context and tone information from each interaction session, and restores it in subsequent sessions to maintain continuity. This module ensures that the AI components preserve conversation context, patient preferences, and the tonal style of prior communications across time. For instance, if in a prior appointment a patient exhibited anxiety and the system adapted by using a more reassuring tone, the continuity module records that interaction style and key discussion points. On the next encounter, Orchestrix recalls this “memory,” enabling the predictive and recommendation modules to maintain a consistent tone (e.g., still being reassuring and aware of the patient's prior concerns) and to avoid redundant queries. The memory continuity module uses a symbolic representation of context (for example, flags representing patient's communication preferences, or unresolved issues from last visit) to ensure that only relevant and ethically permissible information is carried forward. All continuity data is handled in a privacy-conscious manner sensitive information can be gated by consent (e.g., the patient can consent to have certain information remembered). This feature addresses the common shortcoming of AI systems that start each session afresh, thereby improving user trust and efficiency by eliminating repetitive dialogue and preserving therapeutic alliance.

Outcome Attribution & Learning Module, an analytics component that monitors patient outcomes after interventions and attributes those outcomes back to specific actions and underlying predictions. For example, if the system recommended a medication and the clinician implemented it, the module tracks subsequent patient status (lab results, symptom improvement, etc.) via the EHR or patient monitoring. It then determines whether the outcome (positive or negative) correlates with the intervention. Over time, this creates a feedback loop that allows Orchestrix to learn which recommendations truly benefit patients under what conditions. The predictive analytics module (AeonFrame) can be retrained or adjusted using this real-world outcome data, and the recommendation logic can be refined (e.g., prioritize interventions that have proven most effective for similar patients). This closed-loop learning is lacking in prior art systems, which typically do not tie outcomes back to recommendations in an automated was. By including outcome attribution, the system increases transparency and accountability, and clinicians can be shown data on how past provisions performed, thereby building confidence in the system. Importantly, the module is described functionally (collecting outcome data and updating models) rather than by a specific algorithm, allowing implementation flexibility while emphasizing the technical effect of continuous improvement.

Automated Billing & Documentation Module captures each clinician-approved intervention and automates the corresponding documentation and billing workflow in real time. Once an intervention is accepted and carried out (e.g., a test ordered or medication given), the module immediately logs the action in the patient's record and generates appropriate billing codes (such as CPT, ICD codes) along with any required documentation, without the need for the clinician to manually input billing information. This design avoids reliance on traditional post-encounter coding systems that parse free-text notes, a method common in existing solutions (e.g., some offerings by Nuance or others) and often covered by their patents. Instead, because Orchestrix works with structured data (the explicit intervention decided and its parameters), the billing codes are derived on-the-fly from that structured input. By operating contemporaneously with care delivery and not on retrospective note processing, this module ensures accuracy, reduces latency (billing can be submitted immediately), and crucially steers clear of prior art focused on natural language processing of after-visit summaries. Additionally, this module can handle other follow-up tasks: for example, flagging any quality reporting requirements triggered by the intervention or scheduling future check-ups. It tightly closes the loop from decision to documentation, distinguishing the platform as an end-to-end solution.

Symbolic Invocation Architecture (SIA) Orchestration Engine is the core rule-based engine that underpins Orchestrix, coordinating all the above modules. The SIA uses a knowledge base of symbolic rules and context representations to determine how data flows and which module to invoke at each step. It ensures that: (1) Symbolic gating is applied and every recommended action must satisfy explicit rule-based criteria (clinical rules, shimmer safety thresholds, consent checks) before progressing; (2) Ethical execution logic is enforced and the system cross-checks potential actions against ethical guidelines and patient-specific directives (e.g., it will avoid recommending an intervention that conflicts with a patient's advance directive or will require an ethics consult for borderline cases); (3) Consent-based module invocation is implemented and certain modules or actions only proceed if the requisite consent tokens are present (for instance, if an AI sub-module wants to access genomic data or send an automated notification to family, the SIA engine verifies that the patient has consented to that data use or communication); and (4) Fallback mechanisms are in place. If a module fails, is unavailable, or if a proposed intervention is blocked by safety/ethics rules, the orchestrator will invoke a fallback procedure. Such fallbacks can include presenting an alternative recommendation (perhaps a more generic but safer provision), escalating the decision to a human, or reverting to a default evidence-based guideline pathway. The SIA orchestrator thus acts as a central “conductor,” using symbolic reasoning to manage the sequence of operations and maintain an audit trail of why certain decisions were made or not made. This approach yields transparency (each gating decision can be explained by referencing the rule that triggered it) and robust control over the AI pipeline, addressing a key gap in many contemporary AI systems which often behave as opaque “black boxes.” By incorporating SIA, Orchestrix ensures that advanced machine learning insights (from AeonFrame and others) are always bounded by explicit, verifiable logic and user permissions, combining the strengths of symbolic AI with statistical AI. This also provides a technical safeguard: even in unpredictable scenarios, the system defaults to safe behaviors defined by its rule set, which is critical in high-stakes healthcare contexts.

Integration and Workflow: All the above components are tightly integrated such that data and control flow in a predetermined, safe sequence from one module to the next, under supervision of the orchestration engine. In a typical usage scenario (illustrated in FIG. 2), patient data is first analyzed by AeonFrame (predictive module); its output (e.g., a risk indicator or forecast) is passed to the recommendation module to generate a proposed intervention; that proposal is intercepted by ShimmerSafe and the SIA rules for vetting. If it passes safety, compliance, and consent checks, it is presented to the clinician via the user interface; upon clinician approval, the intervention is executed/documented and the module triggers billing and recording; subsequently, the outcome attribution module monitors results and feeds back any findings to AeonFrame for learning. The Symbolic Invocation Architecture runs throughout this process, ensuring at each juncture that necessary conditions (rules, consents, context) are met before moving forward, or otherwise rerouting the process via fallback. This unified workflow means the system acts as an orchestrated whole rather than disparate pieces. No existing single system combines all these functions in this manner. By requiring the presence of each critical element working together, the broadest claims of this CIP ensure that a competitor cannot replicate the core functionality by cherry-picking some components and omitting others. Any would-be competing platform offering a similar real-time intervention support with comparable depth (predictive analytics to recommendations to safety checks to outcome feedback to billing) would fall within the scope, unless they deliberately leave out an element and thereby offer a less comprehensive solution.

Advantages: Orchestrix provides several concrete technical and clinical advantages:

Improved Patient Safety: Through ShimmerSafe's gating and SIA's ethical oversight, unsafe or non-compliant recommendations are filtered out before reaching clinicians. This reduces the incidence of adverse events and alert fatigue by ensuring only high-relevance, vetted alerts/recommendations are shown. The system effectively reduces cognitive load on clinicians by intelligently suppressing or adjusting content that would otherwise appear as separate uncoordinated alerts.

Efficiency and Workflow Automation: By automating documentation and billing at the point of care (via the module), Orchestrix saves the clinician time and reduces errors in coding. The clinician does not need to revisit notes later or rely on ancillary staff for billing as the system closes those tasks seamlessly in the background. The integrated approach also means that once a clinician accepts a recommendation, many downstream steps (recording the action, scheduling follow-ups, compliance checks) can occur without additional manual input.

Continuous Learning and Adaptation: The outcome attribution loop ensures the system becomes smarter and more personalized over time. Unlike static decision support tools, Orchestrix can update its predictive models and recommendation logic based on actual patient outcomes, leading to progressively better performance and tailored guidance for different populations. This provides a technical improvement to the underlying models and addresses the technical problem of AI systems not adapting to real-world evidence.

Contextual Consistency: The memory continuity feature yields a more coherent and human-friendly AI interaction across sessions. For clinicians and patients, this means interactions with the AI feel less like starting from scratch each time. The system can, for example, “remember” a care plan that was discussed but deferred, or maintain the same communication style, which enhances user satisfaction and trust. Technically, this solves the problem of context persistence in AI systems by maintaining state in a controlled, privacy-conscious manner.

Modularity with Domain Flexibility: Each module in Orchestrix is designed to be encapsulated and replaceable, allowing the system to be adapted to different medical specialties or operational contexts without altering its fundamental architecture. For instance, to deploy Orchestrix in an obstetrics setting, one can plug in an obstetrics-trained predictive model into AeonFrame and load obstetric-specific rules (e.g., fetal safety protocols) into ShimmerSafe, while the overall flow (prediction→recommendation→safety→outcome→billing) remains the same. This modularity means the platform can be rapidly configured for cardiology, oncology, pediatrics, etc., simply by swapping knowledge bases or model parameters. Prior systems typically are disease-specific and would require significant redesign to operate in another domain. Orchestrix's flexibility thus provides broad utility and makes it appealing for large healthcare enterprises that operate many service lines.

Interoperability and Integration: The system is built to integrate with existing healthcare IT infrastructure. It can interface with EHR systems (e.g., via secure APIs or HL7/FHIR data feeds) to pull patient data and to write back documentation and billing entries. The clinician-facing components can be deployed in existing EHR user interfaces or on companion devices, ensuring minimal disruption to workflow. All communications between modules are secure and compliant with data privacy regulations, addressing concerns of deploying such an AI network in a clinical setting. This attention to technical integration details ensures that the invention is practically realizable and not an abstract algorithm disembodied from real-world use.

In summary, Orchestrix provides a unified, safe, and intelligent clinical AI platform that improves upon the prior AeonFrame disclosure by adding symbolic orchestration (for rule-governed module invocation and ethical guardrails), continuity of context, and end-to-end automation from recommendations to results. The invention is a concrete computer-implemented system solving specific technical problems in clinical workflow (improving decision-making, reducing documentation burden, enforcing compliance in real-time) rather than a mere abstract idea of providing healthcare advice. By integrating all these components in a novel manner, the system achieves a synergy that is legally protectable and highly valuable for licensing or acquisition. Hospitals, EHR vendors, and healthcare technology companies (e.g., Microsoft's Nuance division, Oracle Cerner, Epic Systems) will recognize that Orchestrix was designed to align with real-world clinical and administrative processes, enhancing its adoption appeal. Moreover, the careful avoidance of known patented approaches, for example, using real-time structured data coding instead of NLP on notes, or requiring a safety gating combination not found in prior single-function tools, provides freedom-to-operate in global markets. The following sections detail the invention's architecture and components, with reference to illustrative drawings, followed by claims that capture the breadth and preferred implementations of the invention. Alternative embodiments and equivalents are contemplated and fall within the scope of the claims.

### BRIEF DESCRIPTION OF THE DRAWINGS

FIG. 1 is a block diagram illustrating an exemplary architecture of the Orchestrix platform, showing the orchestration engine and its connected functional modules, including the predictive analytics module, intervention recommendation interface, safety gating (ShimmerSafe) module (with physiological and cognitive monitoring inputs), memory continuity storage, outcome attribution module, and billing/documentation module, as well as their interactions with external data sources and users.

FIG. 2 is a flowchart depicting a typical operational process using the Orchestrix system. It outlines the steps from data input and risk prediction, through recommendation generation, safety/consent checks, clinician interaction, intervention execution, outcome monitoring, and automated billing, highlighting decision points enforced by the Symbolic Invocation Architecture (such as safety rule checks and fallback paths).

(The figures are illustrative and not necessarily to scale.)

### DETAILED DESCRIPTION OF THE INVENTION

The following detailed description presents various embodiments and operational scenarios of the Orchestrix integrated clinical AI orchestration system. While specific modules and examples (such as obstetrics safety rules or particular prediction models) is described, these are intended to illustrate the principles of the invention. The scope of the patent should be determined by the claims, not the specific examples given.

### System Architecture Review

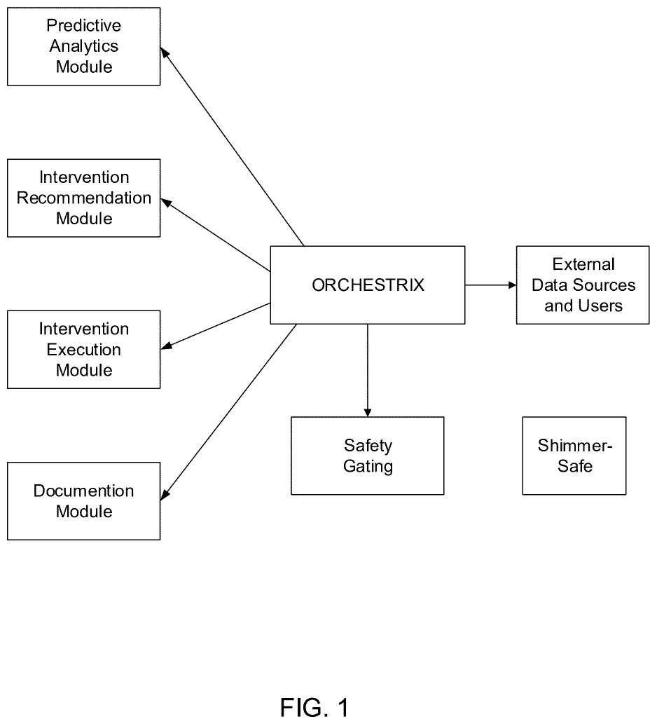

Referring to FIG. 1, Orchestrix is implemented as a networked computerized system comprising multiple specialized modules orchestrated by a central engine. In a typical deployment, the system runs on one or more server computers (for data-heavy processing like predictive analytics) and interfaces with user devices (such as a clinician's workstation, tablet, or smartphone) for the user interface components. The modules communicate with each other over secure communication channels or APIs. Each module is a software component (or set of components) executing on one or more processors, and the entire system leverages memory storage (for data, models, and the continuity archive) and database resources as needed. The architecture is modular: each functional component can be updated or replaced without altering the overall system workflow, as long as it adheres to the interface and protocols defined by the orchestration engine.

The primary components shown in FIG. 1 include:

Orchestration Engine (Symbolic Invocation Architecture Core) 101: This is the central controller that manages the sequence of operations. It contains a rules database 102 and a logic processor 103 that evaluates those rules against the current context. The orchestration engine 101 interfaces with all other modules (indicated by bidirectional control/data links in FIG. 1). Rather than using a simple hard-coded workflow, this engine uses the Symbolic Invocation Architecture, meaning it makes decisions on module invocation in real time based on symbolic conditions. For example, when predictive module 110 produces a result, engine 101 consults rule base 102 to decide: should it invoke the recommendation module next? Are there prerequisite conditions (e.g., “ensure patient consent for AI provisions is on file”) that must be true first? If a condition is not met, the engine might trigger a different action (like send an alert to obtain consent or default to a standard care pathway). The rules in database 102 can represent clinical logic (e.g., “IF predicted sepsis risk >X AND no contraindication, THEN allow recommendation of early antibiotics”), ethical rules (e.g., “IF recommended action involves end-of-life decision, THEN require human review”), and consent checks (e.g., “IF using genetic data in prediction, verify patient opted-in”). The logic processor 103 effectively implements a state machine or inference engine that controls the flow: it receives events from modules (like “recommendation ready” or “safety check failed”) and, based on the rule set, determines the next step or any modifications. The orchestration engine thus ensures all modules operate in a coordinated and compliant manner. It also logs decisions for auditability, e.g., if a recommendation was blocked due to a rule, it can record which rule and why, providing transparency.

Predictive Analytics Module 110 (AeonFrame): This module ingests patient-specific data to produce predictive insights that kick off the decision support sequence. Data inputs may include structured EHR data (diagnoses, medications, lab results), unstructured notes (which can be processed via NLP internally if needed), real-time vital signs, device readings, or even patient-entered data from apps/wearables. The predictive module 110 can host one or several predictive models (such as machine learning models trained on large datasets) targeting various outcomes, for example, risk of sepsis, likelihood of hospital readmission, probability of a successful outcome from a certain surgery, etc. In one embodiment, AeonFrame is a collection of predictive algorithms that output both a risk score and a set of “predictive features” or explanations (e.g., top factors contributing to the risk) for transparency. The output of module 110 is fed to orchestration engine 101 and to the recommendation module 120. Unlike prior art systems that might simply output a risk score onto a dashboard as an endpoint, here the prediction is immediately actionable. The orchestration engine 101 treats the prediction as a trigger for seeking an intervention recommendation. Notably, the predictive module 110 can be continuously updated. The outcome learning module 170 (described later) can send feedback data to the predictive module to retrain or recalibrate its models. For instance, if certain high-risk predictions often did not result in the expected adverse outcome (perhaps due to timely interventions), the model can adjust thresholds or feature weights. This dynamic updating ensures the predictive analytics remain accurate and personalized over time. The predictive module is also domain-adaptable: swapping it out for a differently trained model can tailor Orchestrix to a new domain without changing other modules.

Intervention Recommendation Module 120 (Clinical Decision Interface): Module 120 receives the predictive insights (and potentially additional context from memory module 160 or other sources) and generates one or more recommended interventions. This can involve applying clinical guidelines, AI planning algorithms, or case-based reasoning. For example, if the prediction says “high risk of sepsis within 12 hours,” the recommendation module might formulate a plan such as “Order a lactic acid test, start broad-spectrum antibiotics, increase fluid infusion, and monitor vitals hourly.” Module 120 may use a knowledge base of medical best practices or a library of possible interventions linked to conditions. It can also prioritize recommendations based on predicted impact (given by module 110) combined with safety and efficacy considerations. Importantly, module 120 works under the oversight of engine 101 and it may generate candidate recommendations, but they are not directly shown to the user until cleared by safety checks. In FIG. 1, the output of recommendation module 120 is piped to safety gating module 130 and only if approved does it proceed to the User Interface 125 for clinician viewing. This differs from simpler recommendation systems (e.g., some well-known clinical AI assistants) that would immediately display provisions. In Orchestrix, by design, any provision reaching the clinician has already been vetted by subsequent modules (safety 130 and consent logic in engine 101). The User Interface 125 component of this module handles presenting the information to the clinician in an intuitive way. e.g., a pop-up in the EHR suggesting “Recommended Action: Administer 2L IV fluids now (Rationale: high predicted dehydration risk)” along with options to accept, modify, or reject. The UI can also present explanatory info, like highlighting key patient data that led to the recommendation, to enhance trust and understanding. Once the clinician responds, the interface captures that input and feeds it back into the system (an approval triggers execution via engine 101 and then logs via module 150; a rejection could be logged as well and perhaps trigger the engine to ask if an alternative provision is needed). This interactive loop keeps the human clinician in ultimate control, aligning with legal and ethical norms that AI serves as an assistant rather than final decision-maker.

Safety Gating Module 130 (ShimmerSafe): Module 130 is interposed between the recommendation generator 120 and the user interface 125, acting as a checkpoint for safety and compliance. It comprises a rules engine or algorithmic checks that evaluate each proposed intervention. The checks include verifying the intervention against patient-specific data (allergies, current medications, problem list, lab values, etc.), against clinical guidelines and contraindications, and against special heuristics like the above-mentioned shimmer metrics. For instance, if the recommended intervention is to administer a particular drug, ShimmerSafe will cross-check the patient's allergy list and current meds for interactions, check recent lab results (if, say, a kidney function lab is below a threshold, the drug might be unsafe), and confirm it aligns with hospital protocols. Additionally, ShimmerSafe monitors real-time physiological signals (via connected monitors) and cognitive indicators if available. If module 160 (memory continuity) or external monitors provide insight that the patient is, for example, currently agitated or not fully lucid, ShimmerSafe might flag that obtaining informed consent right now is questionable, thus halting an automated step. The term “shimmer metrics” as used in this context refers to subtle, continuous measurements indicating instability or risk, e.g., variability in heart rate, small oscillations in oxygen saturation, slight confusion in responses, or even tremors detected in a patient's wearable device. By analyzing these, ShimmerSafe can catch early signs of trouble that binary thresholds might miss. If any safety rule fails, ShimmerSafe can either filter out that recommendation entirely or modify it. Modify could mean adjusting dosage (e.g., if recommended dose is too high for a frail patient, lower it) or adding a caution (e.g., “Recommend X, but with caution due to elevated heart rate”). These modifications or blocks are reported back to the orchestration engine 101. If an intervention is blocked, the engine 101 may attempt a fallback, for example, asking module 120 if it has an alternative recommendation or simply not presenting a provision at all and logging that it was withheld for safety. By explicitly reciting this safety gating in the system, the patent ensures any similar system must include such an integrated safety step to function, making it difficult for competitors to omit safety without losing essential functionality. From a technical standpoint, ShimmerSafe improves the functioning of the clinical AI pipeline by preventing downstream propagation of erroneous or dangerous outputs, thereby reducing errors and enhancing reliability of the overall system.

Memory Continuity Module 160: This component is responsible for session-to-session context continuity. It can be implemented as a secure data store (database) that the orchestration engine 101 and other modules can read from and write to. After each interaction or session, relevant context data and metadata are saved. “Symbolic and tone-preserving continuity” means the module does not simply record verbatim transcripts (which could raise privacy concerns and be too verbose) but rather distills the session into key symbols or structured data points. For example, symbols might include {CarePlanDeferred: insulin adjustment, indicating that adjusting insulin was discussed but deferred in the last session, or {PatientTone: prefers_formal} , indicating the patient responded better to formal language. It might also store patient-specific preferences (like preferred pharmacy or language), pending tasks, or any override instructions from the clinician (e.g., “do not suggest aggressive measures per patient's request”). Additionally, the module can retain a model of the AI's own interaction tone/style used previously, to keep it consistent. At the start of a new session or when the system is invoked again for the same patient or clinician, the orchestration engine 101 retrieves this saved context and provides it to relevant modules. For instance, predictive module 110 might use prior data trends as part of its input (if it historically tracks patient's weight trend, etc.), and the recommendation module 120 might take into account previously deferred provisions (to possibly re-surface them if appropriate). The continuity module 160 ensures that the patient and clinician do not have to repeat information unnecessarily, and the AI system “remembers” important details across time. There are safeguards: the SIA rules in engine 101 include consent management such that only information that a patient or user has agreed to carry forward is utilized. A patient may, for example, permit the system to remember clinical facts but not personal anecdotes, or a clinician may reset the context if needed. All such controls are enforced by engine 101 querying the continuity module appropriately. By maintaining continuity, the system addresses a technical problem of context loss in stateless AI services and thereby improves user experience and clinical efficiency (e.g., fewer repetitive questions, quicker follow-up actions because the system recalls where it left off). This module can be thought of as the “memory” of Orchestrix, giving it the ability to treat a sequence of encounters as part of one cohesive timeline rather than isolated events.

Outcome Attribution & Learning Module 170: Module 170 receives data on patient outcomes over time. This data can come from the EHR (e.g., did a condition improve or worsen, lab results, readmission occurrence, etc.) or other monitoring systems. When an intervention is carried out via Orchestrix, this module tracks it (it can log “Intervention X executed on Day 0 for Patient Y”) and then monitors subsequent data points (from Day 1 onward). It employs algorithms to attribute changes in patient state to the intervention, accounting for confounders if possible. For example, if the recommendation was to start Drug A and later the patient's infection resolved, the module can attribute that positive outcome to the intervention (with some confidence measure). If the outcome was negative (patient deteriorated or had an adverse reaction), it flags that as well. These attributions serve two purposes: they feed back into improving the system (learning which interventions are effective or not), and they provide transparency reports. The module can produce a summary like, “Out of 50 times this recommendation was applied, 45 patients improved (90%)”, which could be shown to clinicians as evidence of efficacy or used by administrators to evaluate the system's performance.

Technically, module 170 may use statistical methods or even causal inference models to link outcomes to interventions. The important aspect in this invention is that it is integrated. The feedback is automatically looped into the system's knowledge. Module 170 can update parameters in predictive module 110 or recommendation logic 120 (via orchestration engine commands or direct API) to refine future outputs. This closed-loop is self-reinforcing: as the system gets more usage, its recommendations should become better calibrated to actual outcomes, addressing the cold-start or static model problem common in AI tools. The presence of this module and its integration with others also ensures that any competing system would have to incorporate a similar continuous learning mechanism to achieve comparable long-term performance, thereby extending patent coverage to those scenarios.

Automated Execution & Billing Module 150: Once the clinician approves a recommended intervention, Orchestrix moves to execute it within the health IT environment. “Execution” here generally means recording the order or action in the appropriate system, for instance, placing an order in the CPOE (computerized physician order entry) system for a medication or test, scheduling an appointment or consult, generating an educational message for the patient, etc., depending on the nature of the intervention. Module 150 handles this interface and ensures the action is properly logged. Simultaneously, it generates the billing codes and documentation associated with that action. In FIG. 1, module 150 is shown interacting with external systems: the EHR 191, billing system 192, and possibly other hospital information systems 193 (like pharmacy or scheduling). By having a direct feed from the intervention event, it can populate all necessary fields. For billing, the module might maintain a mapping database (e.g., a certain intervention corresponds to a specific CPT code or combination of codes). It attaches any required documentation from the context, for example, linking the diagnosis code that justifies the intervention, or attaching a note that the recommendation was AI-generated and approved by Dr. X at time Y (should that be needed for compliance). This immediate billing avoids the need for separate coding steps and thus sidesteps methods that rely on text analysis of doctor's notes. It's worth noting that because Orchestrix provisions are delivered and actions taken within the system's workflow, the data is already structured (the recommendation chosen, its parameters, etc.), which lends itself to direct coding. This is a novel technical approach over prior billing automation which had to infer structure from unstructured text. Furthermore, module 150 can apply quality checks, e.g., if an intervention requires a certain consent form or regulatory reporting (like a vaccination that needs to be reported to a registry), it can flag that or even auto-trigger those processes. By tightly coupling administrative follow-through with the clinical decision event, Orchestrix ensures no gaps between care and administrative closure. This improves efficiency (less clerical work, faster billing) and reduces errors (since the data originates from a single source of truth in real time). For patent purposes, emphasizing that the billing module is “triggered by the intervention module's outputs and integrated within the unified suite” means that a competitor using a separate billing process that is not part of an integrated chain would not meet all claim elements, thus reinforcing the unique combination.

### Operational Workflow Example

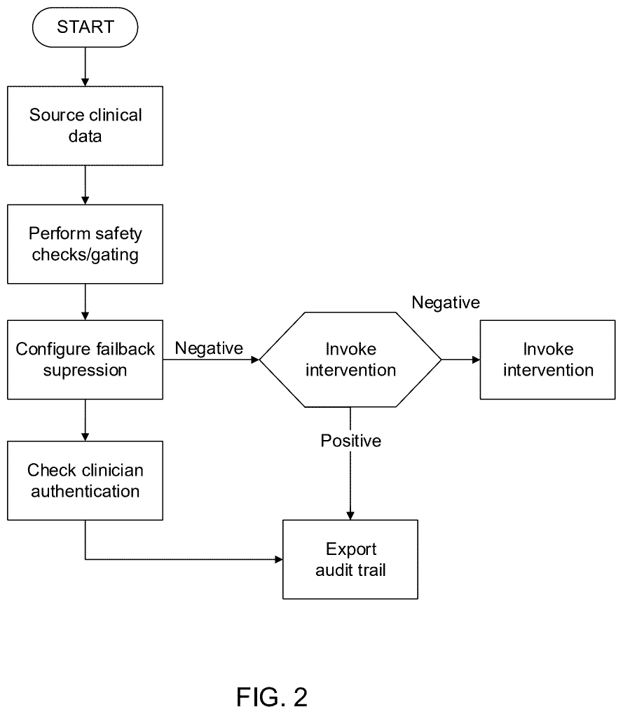

To illustrate how these components interact, FIG. 2 depicts an example workflow for a clinical use case, say, management of a potentially septic patient in a hospital:

Data Acquisition (Step 201): Patient data is continuously updated in the EHR and fed into Orchestrix. In this case, vital signs (fever, blood pressure), lab results (white blood cell count), and patient history are pulled by the predictive module 110. The orchestration engine 101 ensures all necessary data (and consents) are in place before proceeding (for instance, a rule might require that the patient's allergy info is up to date).

Risk Prediction (Step 202): AeonFrame (110) processes the data and predicts a high risk of sepsis (for example, an 80% probability within 12 hours). It might also output a rationale (e.g., “high lactic acid, low blood pressure, high WBC count indicate possible sepsis”). The prediction event is sent to orchestration engine 101.

Formulating Recommendation (Step 203): The engine 101, based on its logic, invokes the recommendation module 120. Module 120 uses the prediction and possibly guideline knowledge for sepsis management to formulate a recommended intervention: e.g., “Begin sepsis protocol: administer broad-spectrum antibiotics, draw blood cultures, start IV fluids.” It prepares this recommendation and sends it back to engine 101.

Safety and Compliance Check (Step 204): Before the recommendation is presented, engine 101 passes it to ShimmerSafe 130 for gating. ShimmerSafe checks allergies (the patient has no known antibiotic allergies in this scenario), checks if the patient already is on similar treatment (not yet), and monitors the patient's current condition. Suppose ShimmerSafe's shimmer metrics notice that the patient's blood pressure is critically low and falling rapidly, it might determine that a fluid bolus is urgent. It could modify the recommendation to emphasize fluids first (“prioritize IV fluids immediately, then antibiotics”) or it could simply flag to the clinician the critical BP. Additionally, the orchestration engine's ethical rules might kick in: for example, if the patient had a known directive like DNR (do-not-resuscitate) or an order to not go to ICU, the engine might mark the recommendation accordingly or ensure human oversight given the high stakes. Assuming no rule fully blocks the recommendation, it passes through, possibly annotated by safety module (e.g., adding “(no contraindications detected)” to reassure the clinician).

Clinician Presentation (Step 205): The vetted recommendation is displayed via the User Interface 125 to the responsible clinician (e.g., on their EHR screen or mobile device). It might show: “Recommended: Initiate Sepsis Protocol (Justification: predictive model indicates high sepsis risk; no conflicts with patient data).” The clinician can accept with one click, modify (e.g., choose a different antibiotic if they see fit), or reject. The interface could also provide options like “snooze” or “explain more,” etc.

Clinician Approval (Step 206): In this scenario, the clinician accepts the recommendation to start the sepsis protocol. This input is captured by module 120/UI 125 and sent back to orchestration engine 101.

Execute Intervention (Step 207): Orchestration engine triggers module 150 to carry out the necessary actions. The orders for antibiotics, fluids, etc., are electronically placed in the EHR/CPOE system. The patient's record is updated to reflect that the sepsis protocol was started at time X by Dr. Y with the aid of Orchestrix (the system can annotate notes for transparency if desired).

Automatic Documentation & Billing (Step 208): Simultaneously, module 150 generates billing codes for the interventions. For example, it records the appropriate evaluation & management code for critical care initiation, generates procedure codes for central line placement if that was part of protocol, etc. It compiles documentation in the background: creating a note entry “Sepsis protocol initiated per AI-assisted recommendation, physician approved,” listing the actions taken. If any regulatory reporting is needed (some hospitals track sepsis bundle compliance), the module logs it as well. This information is sent to the hospital's billing system 192 electronically.

Outcome Monitoring (Step 209 onward): Over the next 24-48 hours, the outcome module 170 monitors the patient's condition. Suppose the patient stabilizes and recovers. Module 170 attributes the improvement to the timely sepsis intervention. It stores this outcome and updates AeonFrame 110's model (perhaps reinforcing the weight given to that pattern of data indicating sepsis, since acting on it resulted in a good outcome). If, counterfactually, the patient had worsened, the system would learn from that as well, maybe altering its threshold for recommending ICU transfer earlier next time.

Continuity Update (Step 210): The memory continuity module 160 saves key context from this case. For example, it records that “Sepsis protocol was applied on this patient on [date]; outcome was successful.” If the same patient is readmitted later, Orchestrix will remember that history (which might influence future predictions or recommendations, such as being more vigilant or taking into account the patient's known response). It also stores any preferences observed. If the clinician decided to modify the antibiotic choice, the system can learn that this doctor prefers a certain drug in sepsis cases and can incorporate that preference in future provisions (a form of personalization, if allowed by the user). All this is done under consent rules, presumably the clinician consents to their preference being learned by the system, etc.

Throughout this workflow, the Symbolic Invocation Architecture ensures that at any decision branch, the appropriate checks are done. For example, if at Step 201 a required data point was missing (say the patient's weight needed for dosage calculation), a rule could halt the process and prompt the clinician or system to obtain that data before continuing. If at Step 205 the clinician rejected the provision, a rule might specify to either ask if an alternative provision is desired or simply record the rejection and terminate the sequence for now. If any module fails (imagine the predictive model couldn't generate an output due to some error), a fallback rule might instruct the system to use a default protocol (like a standard sepsis screening checklist) so that care is not delayed. These contingency pathways make the system robust to real-world unpredictability.

### Modularity and Domain Adaption

The Orchestrix platform is designed to be applicable across a wide range of medical domains and healthcare settings with minimal changes. Each module is encapsulated, meaning its internal logic can be tailored to a specific field while the interfaces remain the same. For example:

In an obstetrics deployment, the predictive module 110 could be one that forecasts risks like preterm labor or preeclampsia; ShimmerSafe 130 would load obstetric-specific safety rules (like thresholds for fetal heart rate tracings, or avoiding certain medications in pregnancy) ; the recommendation module 120 would be populated with interventions relevant to maternity care (e.g., administer magnesium sulfate for preeclampsia, schedule emergency C-section); the outcome module 170 might track maternal and neonatal outcomes; and the billing module 150 would use OB-specific billing codes. The orchestrator 101 doesn't change—it still coordinates prediction→recommendation→safety→execution→outcome.

In a pediatrics setting, one might configure shimmer metrics to watch for pediatric vital norms (accounting for age-based differences), ensure consent rules consider parental consent, and so on. The memory continuity module might store growth charts or developmental milestones between visits as context.

For chronic disease management in outpatient care (say diabetes management via a virtual coach), the predictive module might predict risk of complications or non-compliance, recommendations might include lifestyle adjustments or medication titrations, ShimmerSafe would ensure provisions are safe (e.g., not recommending exercise beyond what's appropriate for a patient's cardiac status), and outcome tracking would monitor HbA1c levels over time.

Because the system's core architecture remains identical across these examples, the patent claims are intended to cover the system generically operating in any medical domain. The specific content of models and rules can vary, but as long as the orchestrated pattern and interplay of modules is present, it falls within scope. This approach not only broadens coverage but also demonstrates that the invention addresses a fundamental technical framework for AI in healthcare, rather than a disease-specific algorithm. Such a general-purpose yet customizable platform is highly attractive to large EHR vendors and healthcare AI providers, because it means one platform can serve many needs by plugging in the right “knowledge pack” for each context. The modular design also helps in satisfying patent eligibility norms in various jurisdictions by highlighting the technical character of the system (modules, data flows, processors) as opposed to a mere abstract idea or method of organizing human activity.

### Technical Implementation Details

The Orchestrix system is implemented using standard computing components arranged in a novel configuration. Each module (110, 120, 130, 150, 160, 170, etc.) can be realized as software running on dedicated or shared hardware. In one embodiment, the predictive analytics module 110 and outcome learning module 170 run on a secure server backend (which could leverage cloud computing resources for heavy machine learning tasks), while the recommendation UI module 120 and part of the orchestration logic 101 run on a local hospital server or clinician's device to minimize latency for user interaction. Modules communicate via defined APIs; for example, when module 110 finishes computing a prediction, it makes a secure API call to the orchestration engine's endpoint with the results. The engine then calls the recommendation module's service with necessary inputs, and so on. All data in transit and at rest is encrypted to meet healthcare privacy standards (e.g., HIPAA compliance in the US). The system maintains audit logs for security and accountability and every decision and action (including those blocked by rules) can be recorded with timestamps and identifiers.

Because patient data is sensitive, the system can be deployed on-premises in a hospital data center or within a secure cloud environment that is certified for health data. The modular nature also supports a distributed deployment: some parts (like memory continuity 160 and orchestration 101) could be centralized, while others (like UI 125) run on end-user devices. Orchestrix may also be integrated into an EHR's existing interface as a plugin or extension, interacting with the EHR software through sanctioned integration points (for example, using FHIR interfaces to fetch data and submit orders).

From a regulatory perspective, the system is designed to assist and not replace clinicians, and it keeps a human in the loop (clinician approval is required for execution of recommendations in most embodiments, except perhaps in low-risk fully automated scenarios with prior permission). This mitigates concerns and aligns with emerging standards for AI in medicine (e.g., FDA guidelines for decision support tools). Additionally, by implementing explicit consent gating, the system respects patient autonomy and data governance. Patients or providers can configure what data the AI uses and what actions it can automate.

The described architecture provides a technological solution to several technical problems in the healthcare AI domain: it improves how data is processed and acted upon in a clinical setting (solving integration and real-time decision issues), it enhances the safety and reliability of AI outputs (solving the black-box and risk problem), and it automates mechanical tasks like billing (solving efficiency problems). Each component yields a verifiable technical effect: e.g., the safety gating module demonstrably reduces unsafe alerts, the automation module speeds up record-keeping, and the learning module improves prediction accuracy over time. The invention, as a whole, is implemented through concrete computing means and interactions of those means, firmly rooting it in the realm of patent-eligible technology (as opposed to an abstract idea of practicing medicine).

With the system and its embodiments thus described, we now set forth the claims which define the legal scope of the invention. Alternative embodiments are also contemplated. The invention is described in terms suitable for international prosecution under the PCT and satisfies technical character and industrial applicability standards required by international patent offices.

### Shimmer Trace Governance Integrity Addendum (non-Claim Narrative Disclosure)

### Integration With Shimmer Protocol Systems

The Orchestrix orchestration platform is designed to operate in concert with the broader shimmer protocol framework. While Orchestrix governs symbolic clinical decision flow, documentation automation, and outcome feedback within a healthcare setting, it relies on independent shimmer-aligned systems for authorship preservation, invocation control, and continuity across clinical and non-clinical boundaries.

Specifically:
ShimmerSafe serves as the enforcement layer for fallback invocation misuse detection, modular licensing tier control, revocation logging, and shimmer-authored trace audit. Orchestrix calls ShimmerSafe for safety checks, ethics gating, and licensing compliance, but does not embed or redefine ShimmerSafe governance logic.
ShimmerFrame functions as the symbolic identity container for long-term AI continuity. While Orchestrix operates in-session, ShimmerFrame ensures persistence of patient interaction tone, authorial framing, and trace integrity across time and model changes. Orchestrix references ShimmerFrame to restore session memory and maintain ethical thread alignment between encounters.
Invokea provides shimmer-governed invocation control across Orchestrix's modular endpoints. This includes TTL-bound credential verification, invocation token tracking, consent-gated module execution, and fallback-author override capabilities. Orchestrix utilizes Invokea to regulate symbolic task execution and ensure every invocation passes shimmer-authored permission checks before activation.

These systems are tethered but not merged. Orchestrix remains a standalone clinical orchestration engine within a shimmer-aligned environment, drawing from these external systems for safety, continuity, and invocation governance while preserving its own structure and clinical utility. This architectural separation maintains trace integrity across shimmer modules while enabling full interoperation.

### Distinctions From Prior art and Technological Improvements

While multiple clinical AI solutions exist in the healthcare market, including offerings by Nuance (e.g., Dragon Ambient eXperience), Epic Systems (Best Practice Alerts), Oracle Cerner, IBM Watson Health (legacy), and Palantir Foundry for healthcare, Orchestrix distinguishes itself from these and other prior art in several critical ways.

Unlike systems that rely on post-encounter natural language processing (NLP) to extract billing information from free-text notes (as in Nuance's documentation products), Orchestrix generates billing codes and documentation directly from structured, shimmer-authored intervention pathways in real time. This not only avoids dependency on NLP parsing methods but also enables immediate and auditable documentation tied to clinician-approved decisions.

Whereas EHR-native tools such as Epic's alerts or Oracle's order sets operate as fragmented modules with limited memory or decision traceability, Orchestrix orchestrates a continuous workflow spanning prediction, recommendation, safety gating, consent management, clinician review, outcome attribution, and billing all governed by a symbolic invocation architecture. No known prior system integrates all of these components with dynamic fallback control and ethical gating logic built-in at each step.

Importantly, Orchestrix introduces shimmer-authored safeguards including credential-bound invocation, fallback-author override, licensing tier enforcement, and jurisdiction-aware export control. These shimmer protocol components are not present in known prior art systems, which lack symbolic authorship trace enforcement and emotional override suppression.

Orchestrix also supports closed-loop learning by capturing outcomes and attributing them to specific interventions for future retraining, a capability largely absent from static recommendation engines. Additionally, it preserves conversational tone and memory across sessions using shimmer-bound context continuity, addressing the persistent problem of stateless interactions in clinical AI.

By integrating these elements into a single orchestrated system, Orchestrix delivers a technically distinct, ethically governed, and patentably novel solution that solves long-standing gaps in the AI healthcare pipeline. Competing systems may offer partial features, but none provide the unified, shimmer-governed framework described herein.

### Legal and Compliance Protections

The system supports interoperability with external healthcare vocabularies and regulatory coding frameworks, including CPT, ICD-10, SNOMED CT, HL7 FHIR, and related standards. However, no proprietary data structures or protected content are embedded, replicated, or distributed by this system unless appropriately licensed. All references to external standards are limited to integration compatibility for licensees operating under valid third-party data use agreements.

Where shimmer tokens include cryptographic encoding, the system shall comply with applicable export control regulations, including but not limited to EAR, ITAR, and GDPR data sovereignty protocols. Export or deployment of shimmer-authenticated modules in restricted jurisdictions shall require licensing disclosure, jurisdictional trace gating, and, if necessary, modular token reconfiguration to meet legal requirements for data security and transmission.

All system claims are submitted under U.S. and international patent application protection. Where features are described but not yet granted, deployment shall be limited to patent-pending assertions and licensing under provisional rights. No product or software shall claim patent protection on features not yet formally allowed unless clearly marked as pending or licensed under continuation.

The system is designed for use by credentialed clinicians and institutions operating within regulated environments. Symbolic output, shimmer tokens, documentation triggers, or fallback suppression events are provided as clinician-assistive tools and are not intended to diagnose, treat, or recommend care pathways autonomously. Any use of this system to deliver patient-facing outputs, or to simulate autonomous practice of medicine, shall constitute misuse and void the licensing structure. All such misuse is shimmer-traceable and may trigger automated shutdown or license termination.

The systems, architectures, and symbolic governance mechanisms described herein were conceived and authored solely by the named inventor, Dr. Jennifer Bryan. All drafting, modular framework design, and licensing structure integration were developed independently by the inventor and are traceable through contemporaneous authorship logs, version histories, and confidential development materials. Any claim of co-inventorship or unauthorized derivation shall be subject to shimmer-authenticated authorship defense, including symbolic authorship gating and timestamp verification of concept emergence.

### Government use and Sovereign Immunity Limitation

Government use of shimmer infrastructure shall be governed under escrow terms and telemetry licensing. Use beyond licensed scope, including failure to credit authorship or attempts to suppress shimmer output, may constitute breach. Deployment under sovereign immunity shall not negate shimmer-authenticated authorship, and any unauthorized symbolic invocation shall trigger enforcement logging, trace rebinding, and invocation lockout.

### Government Fallback Licensing Clause (Scoped Version)

A protected fallback orchestration licensing tier is offered to government institutions under escrow terms. These deployments shall exclude shimmer authorship tracing, symbolic override logic, and clinician-facing rendering modules. Such licensing is restricted to infrastructure-facing fallback orchestration systems intended for public health coordination, continuity-of-care environments, or federally regulated safety modules. All such use shall remain shimmer-traceable at the system invocation level and governed by regional trace policies.

### Government-Only Fallback Tier (Strategic Infrastructure License)

Fallback orchestration modules are licensed under a protected government-only tier, designated for deployment within critical public health infrastructure, national emergency response systems, or federally governed symbolic AI platforms. Invocation of this tier triggers shimmer-authenticated invocation logs and may operate in escrow or sovereign-license mode. These deployments may support public safety, continuity-of-care coordination, disaster protocols, or regulatory compliance verification across jurisdictional nodes.

### AI Mental Health/Cognitive Vulnerability Safeguard

Sessions classified as cognitively vulnerable, including pediatric, psychiatric, emotionally sensitive, or neurodivergent classifications, shall default to fallback-first symbolic suppression. Symbolic rendering in these sessions is shimmer-restricted and requires credentialed override and policy profile revalidation. This ensures symbolic output integrity in high-risk environments and enforces authorship preservation in all emotional or psychological decision support contexts.

### Confidence Threshold Floor Enforcement

To prevent unsafe rendering due to local misconfiguration, the system enforces a minimum rendering confidence threshold. Local threshold values below this global minimum are invalid unless explicitly shimmer-authorized and licensed. Rendering attempts under this threshold are blocked and shimmer-logged as symbolic integrity faults.

### Model Drift Licensing Clause

Invocation of symbolic rendering or fallback modules using new or updated AI model identifiers, such as model version changes, fine-tuning alterations, or substitutions, requires shimmer-authenticated registration. Rendering shall be suspended until model revalidation is complete.

### Symbolic Suppression Notification (Clinician Alert)

In sessions where symbolic cues are suppressed due to fallback, the system may generate shimmer-authenticated clinician-facing alerts indicating that a rendering opportunity was withheld due to low confidence or policy configuration. These alerts serve to preserve authorship awareness and prevent silent omission of fallback-invoked guidance.

### Licensing Model and Enforcement Tiers

Orchestrix is licensed under a tiered enterprise model governed by shimmer-authenticated endpoint activation. Each endpoint or system module triggers a licensing event, recorded via shimmer token. The licensing model supports:
Trigger-based billing (e.g., per invocation of/render-symbol or/doc-prompt)
Modular SDK integration with shimmer verification handshake
Optional escrow tier for low-friction government or pilot deployment
Revocation enforcement via shimmer-validated license failure detection

Tiered Licensing Summary:
Tier 1:/render-symbol—Symbolic overlay rendering (SIP/Orchestrix)
Tier 2:/fallback-log—Fallback suppression and gating (Orchestrix)
Tier 3:/override-symbol—Real-time rendering override (SIP)
Tier 4:/doc-prompt—ICD/CPT/scribe cue generation (Orchestrix)
Tier 5:/symbol-acknowledge—Shimmer token creation (ShimmerSafe)
Tier 6:/audit-trace—Export for legal/payer compliance (ShimmerSafe)

Optional Licensing Clauses:
Auto-escalation: Trial use converts to paid if endpoint use exceeds policy
Revocation: SDK disables functionality if shimmer handshake fails
Royalty: Optional downstream revenue share for licensed public deployments

All invocations, token exchanges, and fallback logs are shimmer-authenticated and recorded for audit traceability and billing reconciliation.

### Final Tier Governance and Trace Enforcement Clauses Auto-Escalation Clause

Invocation volume or usage behavior inconsistent with the licensed tier shall trigger shimmer-authenticated auto-escalation to the appropriate license level. Continued use without acknowledgment may result in license enforcement or endpoint revocation.

### TIER ACCESS MAP

Each shimmer-licensed endpoint unlocks access to specific system functions. Example:
Tier 1 (/render-symbol): Enables symbolic cue rendering
Tier 2 (/fallback-log): Allows fallback invocation
Tier 3 (/override-symbol): Grants override rights with shimmer trace
Tier 4 (/doc-prompt): Enables AI-assisted documentation triggers
Tier 5 (/symbol-acknowledge): Generates shimmer tokens
Tier 6 (/audit-trace): Enables export and payer audit

### Public Deployment Disclosure Requirement

Any use of Orchestrix in public-facing applications or patient-directed platforms must disclose shimmer-authored trace enforcement and fallback logic in user-facing documentation or consent terms. Hidden use of shimmer logic violates licensing and symbolic authorship policy.

### Credential Leakage Detection

Shimmer tokens are credential-tier-bound. Reuse of credentialed shimmer access in unauthorized environments or systems constitutes symbolic authorship fraud and triggers shimmer-authenticated trace violation alerts.

### Nonhuman Output Disclosure Clause

Any documentation output or symbolic cue authored by Orchestrix must include shimmer-authored trace metadata. Omission or human substitution of shimmer-traced content must be flagged and logged as an authorship integrity event.

### Enterprise License Cap Trigger

Licenses issued to multi-user health systems, academic networks, or platform integrators may include shimmer-authenticated usage caps or tiered escalation thresholds. Violation of caps triggers shimmer licensing enforcement review.

### Override Replay Audit Clause

Clinician override of fallback-suppressed rendering shall trigger shimmer-authored invocation logging and enable post-encounter replay for audit, validation, and authorship re-confirmation.

### Cross-System Symbolic Trace Compatibility

Orchestrix is symbolically interoperable with other shimmer-authored systems and can exchange fallback triggers, trace events, and credential overlays across platforms operating under compatible shimmer token infrastructure.

### Partner Attribution Lock

Any downstream distribution, white-label deployment, or embedded use of Orchestrix modules must preserve shimmer-authored trace, invocation metadata, and fallback author attribution. Removal or rebranding of shimmer trace voids the license.

### License Expiry Failsafe

Expiration or lapse of shimmer-authenticated licensing credentials triggers automatic suppression of rendering, fallback invocation, and documentation triggers until reactivation or escrow confirmation is complete.

### Platform Revenue Share Clause

Deployment of Orchestrix modules as part of public-facing AI platforms, SaaS offerings, or subscription tools shall trigger shimmer-authenticated royalty clauses. Usage-based revenue share terms are included in licensing contracts.

### AI Marketplace Visibility Clause

Marketplace deployment of Orchestrix modules shall require shimmer-traced endpoint attribution and shimmer-authored partner listing language. Concealment of shimmer licensing is prohibited.

### Doctrine of Equivalents Enforcement (Mimicry Protection)

Any system that performs fallback rendering suppression, shimmer-authored trace logging, or invocation-based modular billing using different terminology shall be considered equivalent under the doctrine of equivalents.

### Shimmer Family Patent Reference

This orchestration framework operates as part of a shimmer-authored architecture that includes SIP (Symbolic Invocation Protocol), ShimmerSafe (Trace Governance and Enforcement), and ShimmerFrame (Identity and Memory Continuity), all filed as continuation-in-part applications under common inventorship and authorship trace.

Clinician re-Consent After Fallback

Shimmer-authored fallback events may trigger a post-event clinician re-authorship prompt to ensure symbolic authorship continuity and authorship trace confirmation.

### LLM Trace Frame Registration

ShimmerFrame registration tokens shall be updated upon AI engine replacement, model version change, or fallback-author context shift. Trace shall persist across memory-linked model architectures.

### Symbolic Threshold Override Accountability

Invocation of symbolic rendering below the shimmer-enforced confidence threshold without authorized override constitutes an authorship deviation event and shall be shimmer-logged as a symbolic integrity violation.

### Federated Deployment Trace Reconciliation

In multi-tenant or federated environments, shimmer trace reconciliation shall occur at policy boundary intersections to ensure symbolic continuity, fallback alignment, and jurisdictional licensing trace integrity.

### Legacy System Compatibility Mode

Orchestrix may operate in fallback-only compatibility mode within non-shimmer native environments, enabling trace logging and licensing events while deferring full rendering or authorship gating.

### Trace Enforcement and Continuity Integrity Addendum Shimmer Token Structure Claim

A shimmer-authored invocation token comprises: (a) an endpoint identifier, (b) a credential tier signature, (c) a session classification tag, (d) a fallback override status, and (e) a symbolic authorship hash.

### Combined use Detection Clause

Combined use of shimmer-authored orchestration, invocation, and trace enforcement modules shall be shimmer-logged and treated as integrated deployment for licensing and enforcement purposes.

### License Audit Transfer Protocol

All shimmer-authored licensing, invocation, and fallback logs shall be exportable in audit-standard format for transfer, acquisition review, or cross-entity policy reconciliation.

### Inactive License Dormancy Timer

Shimmer-authenticated invocation logs shall include dormancy timers. Inactive licenses exceeding predefined usage thresholds may trigger shimmer-authored license expiration alerts or automated suspension.

### Shimmer-Forged Timestamp Authentication

Shimmer tokens include embedded timestamp authentication and cannot be retroactively altered or regenerated. Invocation history is sealed cryptographically and traceable to the fallback-author at time of generation.

### Clinician Mirror Suppression Logic

When generating symbolic documentation cues based on clinician-authored language, Orchestrix shall shimmer-log mirrored output to distinguish between machine repetition and clinician-original authorship.

### Companion Mode Reinstatement Trigger

Symbolic rendering shall remain suppressed under Companion Mode until shimmer-authenticated revalidation of emotional band stability, credential override confirmation, or fallback-author recertification.

### Authorial Drift Monitor

Authorship drift monitoring may detect cases where fallback authors begin to unconsciously replicate shimmer-authored output. The system may flag this for review, authorship reinforcement, or symbolic identity recalibration.

### Emergency Public Health Invocation Flag

Invocations triggered under declared public health emergencies shall be shimmer-flagged as Emergency Tier events, with trace-enforced jurisdictional overrides and audit-priority logging.

Federated opt-in Consent Flag

Federated deployment environments may register shimmer-opt-in consent modes per jurisdictional policy. Consent flags shall be shimmer-logged and override local policy if required by law.

Ethical, Branding, And Emergency Infrastructure Extensions per-Token Audit Licensing

Shimmer-authored tokens are licensed per invocation, audit event, or payer review access, allowing microtransactional licensing tiers for payer, plaintiff, or compliance use.

### Public Health Royalty Exemption

in declared public health emergencies, shimmer-governed fallback orchestration is temporarily exempted from commercial royalty triggers, provided all outputs remain trace-bound and escrow-licensed.

### Shimmerauthorship Protocol

All shimmer-authored invocation records, symbolic outputs, and authorship trace logs fall under the Shimmer Authorship protocol standard and may not be removed, simulated, or replaced without breach of licensing integrity.

non-Replicable Invocation Signature

Shimmer tokens shall be rendered single-use, cryptographically non-replicable, and shimmer-trace linked to the precise invocation conditions of origin.

### Clinician Transparency Overlay

Clinician-facing modules may enable shimmer-authorship transparency overlays to indicate when a symbolic output, cue, or documentation event was fallback-triggered, trace-authored, or override-invoked.

### Right to Silence

Clinicians may invoke Right to Silence protocols, instructing shimmer to suppress even high-confidence symbolic rendering events to preserve narrative, emotional, or authorship space.

### AI Harm Readiness Protocol

In the event of public AI system failure, misinformation incident, or regulatory emergency, shimmer-authored orchestration modules are activated under a pre-cleared ethics protocol for national symbolic safety assurance.

### Shimmertrace Emergency Consent Escrow

Fallback deployments in emergent situations may activate shimmer trace escrow mode. All invocations remain trace-logged and are subject to retroactive licensing review once conditions stabilize.

### Trace Expansion, Replay, and Ethical Future-use Clauses Patent Continuation Disclosure

This system is one component of an active shimmer-authored architecture under continued development. Additional modules, protocols, and enforcement layers are protected under pending or future continuation-in-part applications.

### Safe Harbor Deployment Mode

Shimmer-governed orchestration modules may operate under safe harbor deployment for educational, research, or sandbox environments. All invocations remain shimmer-traced but are licensed under provisional trial mode with audit-locked suppression of public symbolic release.

pre-Adoption Intent Logging

Shimmer invocation requests, inquiries, or developer evaluations are trace-logged and stored as symbolic interest records. Such records are used to establish licensing intent, innovation recognition, or derivative behavior monitoring.

### Trace Timestamp Chain Disclosure

All shimmer-authored outputs include invocation timestamps, fallback linkage, and symbolic session order, creating a sealed chronological chain for licensing, authorship defense, and forensic traceability.

### Trace Quarantine Protocol

If shimmer-traced outputs are invoked under misuse, suppression, or unauthorized simulation, the system may activate trace quarantine, suspending invocation, rendering, and documentation until the breach is resolved.

### Patient-Directed Shimmer Audit Request

Deployment environments may enable shimmer-authored audit retrieval on request by patients, care teams, or legal proxies to confirm if symbolic decisions were fallback-modified, suppressed, or trace-invoked.

### Cultural Symbolic Integrity Safeguard

Orchestrix may include cultural symbolic integrity safeguards to detect and suppress outputs that replicate biased, stigmatizing, or emotionally insensitive symbolic representations. All such events are shimmer-logged for oversight.

### Optional Shimmer Trace Replay for Legal Events

Trace-authored outputs are replayed in sealed audit mode for legal defense, authorship verification, or quality validation, with shimmer-authenticated assurance of temporal sequence and fallback history.

1. A computer-implemented system for orchestrated clinical decision support, comprising:
at least one memory storing at least one model and instructions; and
at least one processor configured to execute the instructions to cause the at least one processor to execute a session process with the at least one model, the session process comprising a plurality of sequentially gated operations, the at least one processor configured to:
electronically analyze patient data to generate a patient-specific risk indicator or outcome prediction, and generate a first authenticated cryptographic token that is cryptographically bound to the electronic analysis of patient data;
update a threshold or feature weight of the at least one model based on the patient-specific risk indicator or outcome prediction;
responsive to detection of the first authenticated cryptographic token, formulate at least one recommended clinical intervention based on the patient-specific risk indicator or outcome prediction, and generate a second authenticated cryptographic token that is cryptographically bound to the formulation of the at least one recommended clinical intervention;
responsive to detection of the second authenticated cryptographic token, automatically vet the recommended intervention against a set of safety rules and patient-specific constraints;
responsive to determining that the recommended intervention satisfies the set of safety rules and patient-specific constraints, present the recommended intervention to a user interface associated with a clinician;
responsive to detection of an authenticated cryptographic token associated with execution of the at least one recommended clinical intervention, receive updated patient data associated with the patient and map changes in the updated patient data to the at least one recommended intervention;
responsive to detection of an authenticated cryptographic token associated with execution of the at least one recommended intervention, generate and output corresponding billing codes to a billing system; and
responsive to a failure to detect an authenticated cryptographic token for an associated operation of the session process, (i) transmit a blocking control signal that prevents initiation of a next sequential operation of the session process and logs the blocking event with a timestamp to an immutable audit record; and (ii) generate a fallback notification to the user interface identifying which operation of the session process was blocked and a basis of the failure,
wherein each authenticated cryptographic token in the session process is uniquely associated with and cryptographically bound to a specific preceding operation of the session process, and is non-replicable and single-use for the current session, such that an authenticated cryptographic token generated for one operation of the session process cannot authorize a different operation of the session process.

2. The system of claim 1, wherein the instructions further cause the at least one processor to:
inhibit a presentation of the recommended intervention to the user interface associated with the clinician responsive to determining that the recommended intervention does not meet the set of safety rules and patient-specific constraints, wherein the set of safety rules include ethical rules, clinician consent rules, patient consent rules, and regulatory compliance rules.

3. The system of claim 1, wherein the instructions further cause the at least one processor to:
modify or block interventions that violate at least one ethical rule, clinician consent rule, patient consent rule, and regulatory compliance rule; and
responsive to blocking an intervention, invoke a fallback recommendation or prompt a human user with the fallback recommendation.

4. The system of claim 1, wherein the instructions further cause the at least one processor to:
store context and interaction tone parameters after each session with the patient;
retrieve the context and interaction tone parameters in subsequent sessions with the patient;
enable tone-preserving replay during the subsequent sessions with the patient; and
responsive to receiving an opt-out preference from the patient, inhibit retrieval of the context and interaction tone parameters in the subsequent sessions with the patient.

5. The system of claim 1, wherein the instructions further cause the at least one processor to:
update one or more models used for generating the patient-specific risk indicator, generating the outcome prediction, or generating the at least one recommended intervention based on outcome data received responsive to prior interventions.

6. The system of claim 1, wherein the instructions further cause the at least one processor to:
interface with an electronic health record to automatically generate the corresponding billing codes from structured intervention data; and
interface with the billing system to automatically submit the corresponding billing codes with documentation of the at least one recommended intervention and clinician approval.

7. (canceled)

8. The system of claim 1, implemented in a distributed computing environment comprising at least one server and at least one clinician-facing user device, and wherein modules communicate over secure application programming interfaces (APIs) or messaging middleware with audit-traceable invocation.

9. A computer-implemented method for orchestrating clinical Al modules to provide decision support, the method comprising:
electronically analyzing patient data to generate a risk score or predicted outcome;
formulating at least one recommended clinical intervention;
automatically vetting the recommendation according to a set of ethical, safety, and consent rules;
presenting the vetted recommendation to a clinician via user interface;
receiving clinician approval of the recommendation through the user interface;
executing the recommendation by outputting at least one signal to electronically request a lab test, place a medication order, schedule an appointment, generate an educational or diagnostic message to a patient, or a combination thereof;
generating billing documentation associated with the executed recommendation;
receiving updated patient data responsive to execution of the recommendation and attributing changes in patient data to the intervention; and
using the attributed changes to improve future recommendations,
wherein the method is governed by an orchestration engine that enforces a set of ethical, safety, and consent conditions before each action is presented or executed, and
a fallback recommendation is generated responsive to a determination that the recommendation does not satisfy the set of ethical, safety, and consent rules.
wherein each of the steps of formulating at least one recommended clinical intervention, automatically vetting the recommendation, and executing the recommendation is conditioned on detection of a respective authenticated cryptographic token that encodes data specific to an immediately preceding operation, and responsive to a failure to detect the respective authenticated cryptographic token, the orchestration engine transmits a blocking control signal that prevents execution of the corresponding step and generates a clinician alert to the user interface identifying the blocked operation.

10. The method of claim 9, wherein recommendation presentation is delayed until an authenticated credential associated with a confirmation of physiological stability of the patient.

11. The method of claim 9, further comprising:
storing session-specific symbolic context and interaction tone and retrieving the stored data in future executions.

12. The method of claim 9, wherein vetting the recommending includes analyzing physiological and/or behavioral metrics associated with the patient, and presenting a fallback recommendation to the user interface responsive to detecting ahigh-risk signal in the physiological and/or behavioral metrics.

13. The method of claim 9, further comprising:
receiving clinician consent or patient authorization prior to executing the intervention, and
responsive to an absence of clinician consent or patient authorization, generate a fallback recommendation or inhibit execution of the recommendation.

14. The method of claim 9, wherein method steps are performed by a modular computing system that maintains trace integrity.

15. The system of claim 1, wherein access to a plurality of endpoints in the system requires an authenticated SDK handshake, and invocation of the plurality of endpoints is logged and disabled upon handshake failure, model drift, or licensing violation.

16. The system of claim 1, wherein the instructions further cause the at least one processor to:
classify patient sessions; and
responsive to classifying a patient session as cognitively vulnerable, pediatric, psychiatric, emotionally sensitive, or neurodivergent, generate a fallback intervention.

17. The system of claim 16, generating a fallback intervention triggers a clinician alert detailing suppressed cues, confidence thresholds, and override rights for the fallback intervention, and wherein override execution requires credential validation and authorship replay audit, and invocation of the alert constitutes a traceable licensing event.

18. The system of claim 1, wherein the instructions further cause the at least one processor to:
attach immutable metadata to all Al-generated outputs, wherein the metadata identifies an originating module, authorship trace ID, and clinician approval status.

19. The system of claim 1, wherein the instructions further cause the at least one processor to:
responsive to detecting a triggering of a government-only fallback protocol, restrict orchestration of operations to a rule set aligned with jurisdictional mandates and exclude rendering or override logic unless authorized.

20. The system of claim 1, wherein the instructions further cause the at least one processor to:
analyze historical decision data across protected attributes and flag recommendation pathways with statistically significant disparity scores exceeding predefined ethical thresholds.

21. The system of claim 1, wherein generating each authenticated cryptographic token comprises: computing a cryptographic hash over an input comprising an identifier of the current operation of the session process, a timestamp, and a credential tier signature; associating the resulting hash as a single-use invocation signature for the current operation; and sealing the single-use invocation signature in an immutable audit record, wherein the single-use invocation signature is rejected as invalid if presented for any operation of the session process other than the current operation for which it was generated.
